(19) United States
(12) Patent Application Publication
(10) Pub. No.: US 2025/0032124 A1
(43) Pub. Date: Jan. 30, 2025

(54) EMBOLIZATION SYSTEM

(71) Applicant: Clearstream Technologies Limited, Enniscorthy, County Wexford (IE)

(72) Inventors: Karen Redmond, Enniscorthy (IE); Fearghal O'Connor, Gorey (IE); Allan Ronan, Enniscorthy (IE); Artur Spodobalski, Blackwater (IE); Piaras Griffin, Naas (IE)

(21) Appl. No.: 18/696,804

(22) PCT Filed: Sep. 30, 2021
(86) PCT No.: PCT/EP2021/076993

§ 371 Date: Mar. 28, 2024

(51) Int. Cl. A61B 17/12; A61B 90/00; A61M 25/10

(52) U.S. Cl. CPC A61B 17/12172; A61B 17/12031; A61B 17/12109; A61B 17/12136; A61B 17/12177; A61B 2017/12095; A61B 2090/3966; A61M 25/10186

(57) ABSTRACT

An embolization system for promoting clot formation in a body lumen (700). The system comprises a self-expandable embolization device (100), having a collapsed delivery configuration and an expanded deployed configuration in which the self-expandable embolization device (100) is radially expanded to anchor to a bodily lumen (700). The system also comprises an inflatable body (800) connected to the embolization device (100), the inflatable body (800) having a deflated configuration and an inflated configuration, wherein in the inflated configuration the inflatable body (800) anchors to the bodily lumen (700). The inflatable body (800) is positionable relative to the self-expandable embolization device (100) such that it is inflatable from the deflated configuration to the inflated configuration as the self-expandable embolization device (100) remains in the collapsed delivery configuration.

### TECHNICAL FIELD

The present disclosure relates to an embolization system comprising a self-expandable embolization device for promoting clot formation in a bodily lumen having a collapsed delivery configuration and an expanded deployed configuration, and a connected inflatable body for pre-positioning the embolization device.

### BACKGROUND

Embolization devices may be deployed in the vasculature at a particular location by a medical practitioner so as to promote blood clot formation and to occlude the blood vessel. Typically, an embolization device may be pushed through a guiding delivery catheter, which has been positioned in the blood vessel, using a delivery wire until the embolization device reaches the distal tip of the delivery catheter. The device is then deployed from the tip of the delivery catheter, for example by pinning (holding static) the delivery wire and retracting the delivery catheter.

The embolization device and the delivery catheter are not anchored to the bodily lumen until the embolization device is deployed. Accordingly, movement of the delivery catheter or the embolization device away from the optimal position within the bodily lumen is possible before or during deployment of the embolization device, making the positioning of the embolization device prone to error. Once the embolization device is deployed, it may be difficult to re-position the embolization device without risking damage to the blood vessel or to the embolization device.

Accordingly, there is a need to reduce the risk of errors in positioning the embolization device during deployment of the embolization device.

### SUMMARY

According to a first aspect of the present disclosure, there is provided an embolization system for promoting clot formation in a body lumen, comprising: a self-expandable embolization device, having a collapsed delivery configuration and an expanded deployed configuration in which the self-expandable embolization device is radially expanded to anchor to a bodily lumen; and an inflatable body connected to the embolization device, the inflatable body having a deflated configuration and an inflated configuration, wherein in the inflated configuration the inflatable body anchors to the bodily lumen; wherein the inflatable body is positionable relative to the self-expandable embolization device such that it is inflatable from the deflated configuration to the inflated configuration as the self-expandable embolization device remains in the collapsed delivery configuration. The inflatable body allows the embolization system to be pre-positioned and anchored within the bodily lumen before deployment of the embolization device. Accordingly, the position of the embolization device relative to the bodily lumen (rather than relative to a delivery catheter which may move relative to the bodily lumen) is controlled. The inflatable body may be deflatable and re-inflatable for re-positioning of the system in the bodily lumen.

The self-expandable embolization device may comprise a core and a plurality of flexible bristles extending at least radially outwardly from the core, the flexible bristles having a collapsed delivery configuration and an expanded deployed configuration in which the bristles extend at least radially outwardly from the core to anchor the embolization device in a bodily lumen. Alternatively, the self-expandable embolization device may comprise a self-expandable layer, the self-expandable layer comprising a plurality of braided strands, the plurality of braided strands having a collapsed delivery configuration and an expanded deployed configuration in which a plurality of the braided strands anchor the embolization device in a bodily lumen, and in which a plurality of the braided strands extend across the bodily lumen to occlude the bodily lumen.

The embolization device and the inflatable body may be slidably connected or non-slidably connected to each other.

The embolization may comprise a sealing mechanism actuatable to fluidically seal the inflatable body in the inflated configuration. The inflatable body may then assist the embolization device in occluding the vessel.

The sealing mechanism may comprise a one-way valve configured to prevent fluid from exiting the inflatable body. Alternatively, the sealing mechanism may comprise a resistive element and a meltable material, wherein an electrical current applied to the resistive element melts the meltable material to seal an inflation port of the inflatable body. Alternatively, the sealing mechanism may comprise a flexible tubular material having an open configuration in which fluid may enter the balloon via a lumen defined by the flexible tubular material, wherein the flexible tubular material is twistable into a closed configuration in which the inflatable body is fluidically sealed.

The embolization device may be configured to be connected to a delivery element via a detach mechanism, the delivery element for delivering the embolization device through a delivery catheter. The detach mechanism may comprise a male or female screw thread on the embolization device or the detach mechanism may comprise an electrolytic element connecting the embolization device to the delivery element.

The inflatable body may be fixed relative to the embolization device and the embolization system may be configured to be connected to a single delivery element via a detach mechanism, wherein the delivery element is for delivering both the embolization device and the inflatable body through a delivery catheter. The detach mechanism may include a sealing mechanism actuatable to fluidically seal the balloon in the inflated configuration.

The embolization device may comprise a stem and a plurality of flexible bristles secured to the stem and extending at least radially outwards from the stem, and wherein the stem defines an inner lumen fluidically connected to the interior of the inflatable body. The stem may be both a securing element for the bristles and as an inflation lumen for the inflatable body.

The embolization device may comprise a device lumen extending longitudinally through the embolization device, wherein the device lumen receives an inflation tube for inflating the inflatable body.

The embolization device and the inflatable body may be slidably connected and the inflatable body may be configured to fit partially or wholly within the device lumen in the deflated configuration. This allows for a more compact delivery configuration of the embolization system.

The inflatable body may comprise an inner lumen extending longitudinally through the inflatable body and configured to receive a delivery element for delivering the embolization device. The embolization device may be configured to fit partially or wholly within the device lumen in the collapsed configuration. This allows for a more compact delivery configuration of the embolization system.

The embolization system may further comprise a sheath slidably received in the inner lumen, wherein the embolization device fits inside the sheath in the collapsed delivery configuration.

The inflatable body may comprise one or more radiopaque markers. The embolization device may comprise one or more radiopaque markers.

### BRIEF DESCRIPTION OF THE DRAWINGS

To enable better understanding of the present disclosure, and to show how the same may be carried into effect, reference will now be made, by way of example only, to the accompanying schematic drawings, in which:

FIG. 1A shows a side view of a self-expandable embolization device for use in an embolization system according to one or more embodiments;

FIG. 1B shows a cross-sectional side view of another self-expandable embolization device for use in an embolization system according to one or more embodiments;

FIG. 2A shows a side view of a self-expandable embolization device for use in an embolization system according to one or more embodiments;

FIG. 2B shows a longitudinal view of the self-expandable embolization shown in FIG. 2A;

FIG. 2C shows a side view the self-expandable embolization device of FIG. 2A in a collapsed configuration;

FIG. 3A shows a cross-sectional side view of an embolization system according to one or more embodiments;

FIG. 3B shows a cross-sectional side view the embolization system of FIG. 3A in another configuration;

FIG. 3C shows a cross-sectional side view the embolization system of FIG. 3A in another configuration;

FIG. 3D shows a cross-sectional side view the embolization system of FIG. 3A in another configuration;

FIG. 4A shows a cross-sectional side view of a detach mechanism for use in an embolization system according to one or more embodiments;

FIG. 4B shows a cross-sectional side view of a detach mechanism for use in an embolization system according to one or more embodiments;

FIG. 5 shows a side view of an embolization system according to one or more embodiments;

FIG. 6A shows a cross-sectional side view of a sealing mechanism for use in an embolization system according to one or more embodiments;

FIG. 6B shows a cross-sectional side view of a sealing mechanism for use in an embolization system according to one or more embodiments;

FIG. 6C shows a cross-sectional side view of a sealing mechanism for use in an embolization system according to one or more embodiments;

FIG. 6D shows a cross-sectional side view of the sealing mechanism of FIG. 6C in a closed configuration;

FIG. 7A shows a cross-sectional side view of an embolization system according to one or more embodiments;

FIG. 7B shows a cross-sectional side view the embolization system of FIG. 7A in another configuration;

FIG. 7C shows a cross-sectional side view the embolization system of FIG. 7A in another configuration;

FIG. 7D shows a cross-sectional side view the embolization system of FIG. 7A in another configuration;

FIG. 8 shows a cross-sectional side view of an embolization system according to one or more embodiments;

FIG. 9A shows a cross-sectional side view of an embolization system according to one or more embodiments;

FIG. 9B shows a cross-sectional side view the embolization system of FIG. 9A in another configuration;

FIG. 9C shows a cross-sectional side view the embolization system of FIG. 9A in another configuration; and

FIG. 10 shows a cross-sectional side view of an embolization system according to one or more embodiments.

### DETAILED DESCRIPTION

Throughout this disclosure the term ‘embolization device’ may refer to a device which may be permanently or semi-permanently implanted in a bodily lumen to promote occlusion of the bodily lumen to embolize the bodily lumen. Accordingly, the ‘embolization device’ may be configured to be disposed within the bodily lumen for a period of time, such as a number of days, or disposed in the lumen indefinitely, to promote occlusion of the lumen. To this end, the ‘embolization device’ may be configured to be selectively detached from a delivery element so that it may be implanted in the bodily lumen in isolation.

Throughout this disclosure the term ‘bodily lumen’ or ‘body lumen’ may refer to the inside space within a tubular structure of the human or animal body. The ‘bodily lumen’ may be, for example, an artery or vein.

Throughout this disclosure the term ‘collapsed delivery configuration’ of an element may refer to a configuration of the element which has a smaller radial extent than an expanded deployed configuration of the element.

Throughout this disclosure the term ‘to anchor’ may refer to partly or fully securing an element in a position.

In some embodiments, a self-expandable embolization device may comprise bristle segments having a core and a plurality of flexible bristles extending radially outwardly of the core. The core may be a stem. The term ‘stem’ may refer to an elongate element which extends longitudinally along the length of the embolization device or bristle segment to act as a backbone for the device or segment, and has a significantly smaller radial extent than the further elements of the embolization device (for example, the plurality of flexible bristles). The stem may extend along substantially the whole longitudinal extent of the plurality of flexible bristles of the device or segment (e.g. when the embolization device is in an unrestrained configuration, collapsed delivery configuration and/or expanded deployed configuration). The stem may extend along substantially the whole length of the embolization device. The bristle segments of each bristle section may be formed on a single core or may be formed on separate connected cores.

The term ‘bristle’ may refer to an elongate strand of material formed substantially a single piece. The ‘bristle’ may be a resilient bristle. The resilient bristle may be biased towards a particular curvature.

Throughout this disclosure, the term ‘radially outwardly’ does not exclude the element additionally extending in the longitudinal direction of the device. For example, the element may extend radially outwardly and longitudinally.

Through this disclosure, the term “radial profile” may refer to a radial extent in a particular direction transverse to a longitudinal direction, for example an axis defining the direction of a delivery catheter or the bodily lumen. For example, the embolization device has a lower radial extent in the collapsed delivery configuration than in the expanded deployed configuration.

Throughout this disclosure, the term “bristle segment” may refer to a group of bristles wherein the spacing between adjacent bristles is less than a predetermined distance. When two bristle segments are “spaced apart”, the spacing between the bristle segments (i.e. the spacing between the most distal bristle of the first segment and the most proximal bristle of the second segment) is greater than the spacing between adjacent bristles within at least one of the bristle segments.

For devices comprising a plurality of bristles, the plurality of flexible bristles may have a collapsed configuration in the collapsed delivery configuration. The plurality of flexible bristles may have an expanded configuration in the expanded deployed configuration. The plurality of bristles may extend radially outwardly from the core or stem in a plurality of circumferential directions about the core or stem.

In the expanded configuration, the plurality of flexible bristles may be configured to anchor the device in the bodily lumen. The plurality of flexible bristles may be configured to provide substantially all of the anchoring force for the embolisation device or the bristle segment in the bodily lumen.

In the expanded configuration, the plurality of flexible bristles may be configured to contact the bodily lumen.

The device adopts the collapsed delivery configuration when the device is positioned inside the delivery catheter. More particularly, in the collapsed delivery configuration, device has a radial extent which is less than the radial extent of the device in the expanded deployed configuration.

The embolization devices disclosed herein may comprise one or more flow restrictors disposed on the device. The flow restrictor may be placed at any point on the device.

For devices comprising a plurality of bristles, the bristles may be made of a flexible or resiliently deformable material such as stainless steel, Elgiloy or Nitinol. Other suitable materials may also be used, such as any suitable polymer or any other shape memory metal or metal alloy.

The flow restrictors disclosed herein may be a thin film membrane made of a self-expanding material such as a polymer, stainless steel or Nitinol.

For devices comprising a core or stem, the core or stem may be made of stainless steel or other suitable material and may comprise a twisted wire from which the bristles extend and on which a flow restrictor is mounted. The stem may alternatively comprise a hollow tube wherein the walls of the hollow tube hold the radially extending bristles in place. For example, the tube may be formed from a shrinkable material. Alternatively or additionally, the bristles may be held by the stem using other means such as adhesives.

The diameter of an individual flexible bristle may range from 0.036 mm (0.0014 inches) to 0.053 mm (0.0021 inches). For example, the diameter of an individual flexible bristle may be 0.0381 mm (0.0015 inches), 0.0445 mm (0.00175 inches) or 0.0508 mm (0.002 inches). The average radial diameter of the radial profile formed by the expanded flexible bristles may range from 5 mm to 30 mm.

A flow restrictor may be a membrane made from thin film Nitinol, thin film PTFE, a thin film elastomer such as polyurethane or any other type of suitable biocompatible material. The membrane may have a thickness of 4 μm to 35 μm and a radial diameter of 5 mm to 20 mm. For example, the diameter of the membrane may be 6.5 mm, 9 mm or 16 mm. Furthermore, the membrane may be non-permeable or semi-permeable.

The embodiments disclosed herein related to an embolization system which comprises a self-expandable embolization device and an inflatable body, wherein the inflatable body is positionable relative to the self-expandable embolization device such that it is inflatable from the deflated configuration to the inflated configuration as the self-expandable embolization device remains in the collapsed delivery configuration. In other words, the embolization inflatable body is positioned, or movable to a position, where the outer surface does not abut against or otherwise cause the embolization device to be deployed by the inflation of the inflatable body.

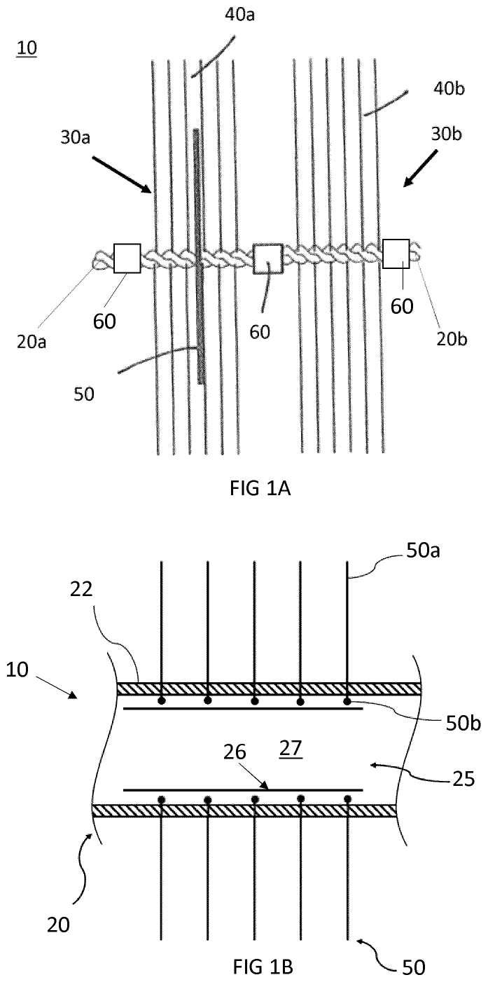

FIG. 1A shows a self-expandable embolization device 10 in an unconstrained configuration. The embolization device 10 comprises a plurality of flexible bristles 40a, 40b having an expanded deployed configuration and a collapsed delivery configurations. The device may comprise a series of segments (for example, two) wherein the bristles 40a of at least one segment 30a are configured to point distally when deployed in a bodily lumen, and the bristles 40b of at least one segment 30b are configured to point proximally (i.e. the opposite direction to the bristles 40a) when deployed in a bodily lumen. In some cases, there is only a proximal segment 30a and distal segment 30b. The bristles of each segment 30a, 30b extend radially outwardly from a core, and more specifically stems 20a, 20b.

A proximally pointing segment is defined as a segment in which the bristles point proximally. The proximally pointing segment 30a may comprise a flow restrictor 50. The flow restrictor may be a cone-shaped membrane open at the proximal end or may be another shape such as disc shaped. The flow restrictor 50 comprises a hole (not shown) through which the stem 20a passes such that the flow restrictor 50 is mounted on the stem 20a. A distally pointing segment is defined as a segment in which the bristles point distally. The flow restrictor 50 may alternatively be present on the distally pointing segment and may be a cone-shaped membrane open at the distal end or may be another shape such as a disc shape.

In some examples, at least one segment, such as in the illustrated case the proximal segment 30a, incorporates a flow restrictor 50. The flow restrictor 50 is configured to restrict blood flow in the vessel when the device 10 is deployed.

In some examples one or more radiopaque markers divides the proximally pointing segment 30a and the distally pointing segment 30b, such as one or more of the radiopaque markers 60 shown in FIG. 1A. The radiopaque marker 60 may alternatively be positioned proximally to the most proximal segment or distally to the most distal segment, or in between segments. The device may comprise one or more markers 60. There may be a proximal marker (not depicted) positioned proximal to the proximal bristle segment 30a, a distal marker (not depicted) distal to the distal segment 30b, and/or an intermediate marker 60 positioned between the proximal bristle segment and the distal segment. In other examples, the device does not comprise any markers.

In some examples the bristles 40a, 40b are formed on respective stems 20a, 20b. In the illustrated example the stems 20a, 20b comprise a twisted metal wire which securely holds the bristles in the twists of the wire. The stems 20a, 20b may alternatively comprise a hollow tube wherein the walls of the hollow tube hold the radially extending bristles in place, as shown in FIG. 1B. For example, the tube may be formed from a heat shrinkable material or a meltable material. Alternatively or additionally, the bristles may be held by the stem using other means such as adhesives.

The stems 20a, 20b may be separately formed and joined by an articulating, flexible or non-articulating joint. For example, in the illustrated example the stems 20a, 20b are connected via the radiopaque marker 60 which is securely fastened to the stems 20a, 20b by crimping, welding, adhesive or the like and which is made of an inflexible material. Alternatively, the marker 60 may merely be a radiopaque band situated at a location on one of the stems or the joint, and the stems 20a, 20b may be joined by an articulating joint, a flexible joint or an inflexible joint. In other examples, the bristle segments may be formed on a single stem and the stem may be more or less flexible at the section joining the bristle segments.

In one case the embolization device comprises only a single proximal segment 30a and a single distal segment 30b. The proximal segment 30a and the distal segment 30b in one case are mounted on a single common stem. The stem 20a of the proximal segment 30a and the stem 20a of the distal segment 30b may form parts of the same continuous stem.

In the case where the device comprises more than two segments, the connection between the two most proximal segments may be stiffer than the distal connections. The distal connections may generally comprise a hinge.

In one example, a flow restrictor 50 comprising a flexible membrane is present in at least one of the segments. The membrane may comprise a disc of thin film material. The flexibility of the membrane means its orientation is controlled by the orientation of the adjacent bristles—in the illustrated example, if the adjacent bristles 40a are forced to point distally the membrane will adjust its configuration accordingly. Thus, if the membrane is deployed from a collapsed condition, such as from within a catheter, the bristles will cause it to open up to an expanded configuration. The membrane may also be placed proximal or distal to the segment.

Optionally, the membrane may comprise lines along which the membrane preferentially collapses when it is in the collapsed configuration, so that folding of the membrane in the collapsed configuration is controlled in a predictable manner.

In one case, the embolization device 10 comprises one or at least two segments. In one configuration the flow restrictor 50 is in the most proximal segment. This is shown in an unconstrained state schematically in FIG. 1A. In the configuration shown the flow restrictor 50 is located within the proximal segment 30a with bristles 40a both proximal and distal to the flow restrictor 50. In some cases, there may be a distal flow restrictor (not shown), and there may be any number of flow restrictors provided on the embolization device.

The flow restrictor 50 may have an outer dimension which is less than an outer dimension of the plurality of anchoring bristles. The flow restrictor may be connected to the stem. In some examples the flow restrictor may have a central hole that is an interference fit on the stem. The central hole in the flow restrictor is preferably smaller than the stem on which it is mounted. The central hole in the flow restrictor may have a diameter which is smaller than the diameter of the stem prior to assembly of the device.

The device 10 has a collapsed configuration to facilitate delivery through a catheter. By placing the flow restrictor 50 within the bristles 40a of the segment 20a, i.e. with bristles proximal and distal to it, it is protected from damage while the implant is being collapsed or pushed through a catheter. Furthermore, any friction between the catheter and the flow restrictor 50 is reduced.

FIG. 1B shows a cross-sectional side view of the self-expandable embolization device 10 comprising a stem 20 and flexible bristles 50 secured to the stem 20 and extending radially outwards from the stem.

The stem 20 of the embolization device comprises a tube having a tube wall 22 defining a lumen 25.

Each of the plurality of flexible bristles 50 penetrates through the tube wall 22 such that a portion 50b is disposed within the lumen 25 of the tube. A free portion 50a of each of the plurality of flexible bristles 50 extends radially outwardly from the tube, and, in particular, the tube wall 22.

As shown in FIG. 1B, optionally the portion 50b disposed within the lumen 25 of the tube is thicker than the free portion 50a (in other embodiments the thickness may be the same). The portion 50b may be configured such that the flexible bristle is secured to the tube. Specifically, the portion 50b may have a larger radius than the radius of a hole in the tube wall 22 through which the flexible bristle passes.

Additionally or alternatively, the portion 50b disposed within the lumen 25 of the tube may comprise a rough portion. The rough portion may be rougher than some or all portions of the free portion 50a. The rough portion may for example comprise a coating which has a higher frictional coefficient than the free portion 50a.

Additionally or alternatively, some or all of the portions 50b may be clamped between an inner mandrel 26 disposed within the lumen 25 of the tube and the tube wall 22, the mandrel 26 defining an inner lumen 27.

The stem 20 and flexible bristles 50 of the embolization device may be manufactured by creating, for example, by machining, a plurality of holes in the tube wall 22 of the tube. One of the plurality of flexible bristles 50 may be inserted through each of the plurality of holes in the tube wall 22 such that a portion 50b of each of the flexible bristles 50 extends into the lumen 25 of the tube.

For example, a guide may be inserted into the lumen 25 of the tube such that the free portion 50a may be guided from the inside of the lumen 25 through the hole to the outside of the tube.

Additionally or alternatively, the tube may have a thin slit along at least a portion of its length so as to facilitate insertion of the flexible bristles 520 through their respective holes from the inside of the lumen.

The embolization devices 10 shown in FIGS. 1A and 1B are removably connectable, via a detach mechanism (not shown), to a delivery element (such as a delivery wire or pusher) for pushing the embolization device 10 through a delivery catheter. The detach mechanism may be any of the detach mechanisms disclosed herein, for example with respect to FIGS. 4A and 4B.

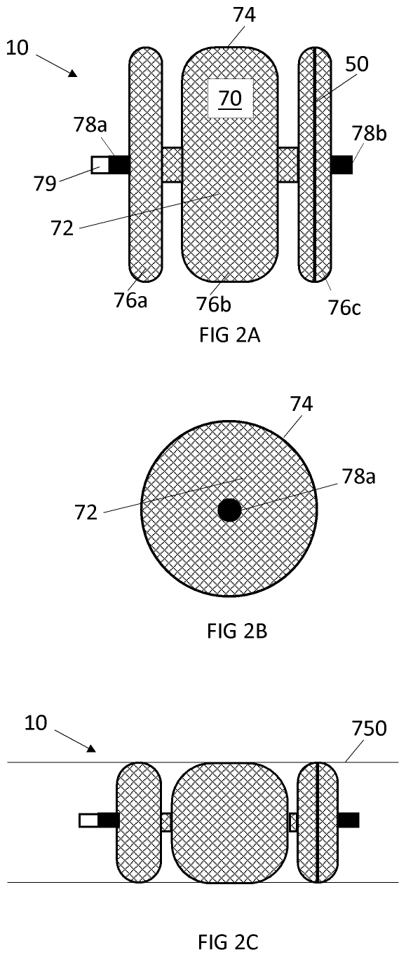

FIG. 2A shows a side view of a self-expandable embolization device 10. The self-expandable embolization device 10 comprises one or more self-expandable layers 70. The self-expandable layer comprises a plurality of braided strands 72, wherein the plurality of braided strands have a collapsed delivery configuration and an expanded deployed configuration in which a plurality of the braided strands 72 anchor the embolization device in a bodily lumen (for example, an outer portion 74 of the layer 70 contacts the wall of the bodily lumen in the deployed configuration). In FIG. 2A, the embolization device is shown in an expanded, unconstrained configuration. The embolisation device 10 is radially compressible to fit inside a delivery catheter in a collapsed delivery configuration. For example, the embolization device is configured to fit inside delivery catheter 750 in a collapsed delivery configuration as shown in FIG. 2C. In the expanded deployed configuration, a plurality of the braided strands 72 extend across the bodily lumen to occlude the bodily lumen. For example, as shown in FIG. 2B, which shows a longitudinal view of the embolization device 10 shown in FIG. 2A, the braided strands 72 extend between points the radial outer portion 74 to form an occluding mesh which generally extends across the bodily lumen to occlude the lumen.

The embolization device 10 may comprise one or more lobes 76a, 76b, 76c. The embolization device 10 may also comprise a flow restrictor 50 configured to restrict blood flow in the vessel when the device 10 is deployed. The flow restrictor may be a disc-shaped or cone-shaped membrane and may be situated inside one of the lobes (for example the membrane may be coupled to the lobe by adhesive or welding). Alternatively, the flow restrictor 50 may be located between one of the lobes 76, or proximal or distal to the lobes 76.

The plurality of braided strands 72 may be fixed at one or both proximal/distal fixing elements of the device 78a, 78b. For example, the braided strands 72 may be clamped, adhered, welded or the like to the proximal/distal fixing elements 78a, 78b to prevent unravelling of the braided strands 72.

The braided strands 72 may be made of a material that is resiliently deformable, such as a metal (e.g. nitinol) or polymer. The braided strands 72 may be made of a material that can be heat treated to set a pre-set shape (such as the shape shown in FIG. 2A). The braided strands 72 may be made of a shape memory alloy such as Nitinol.

The embolization device 10 is removably connectable, via a detach mechanism 79, to a delivery element (such as a delivery wire or pusher) for pushing the embolization device through a delivery catheter. The detach mechanism 79 may be any of the detach mechanisms disclosed herein, for example with respect to FIGS. 4A and 4B.

The embolization device 10 of FIG. 2A may further comprise a lumen (not shown) extending through device 10 from a proximal end to a distal end of the device 10 (e.g. longitudinally through the detach mechanism 79, the proximal and distal fixing elements 78a and 78b, the self-expandable layer 70 and the flow restrictor 50).

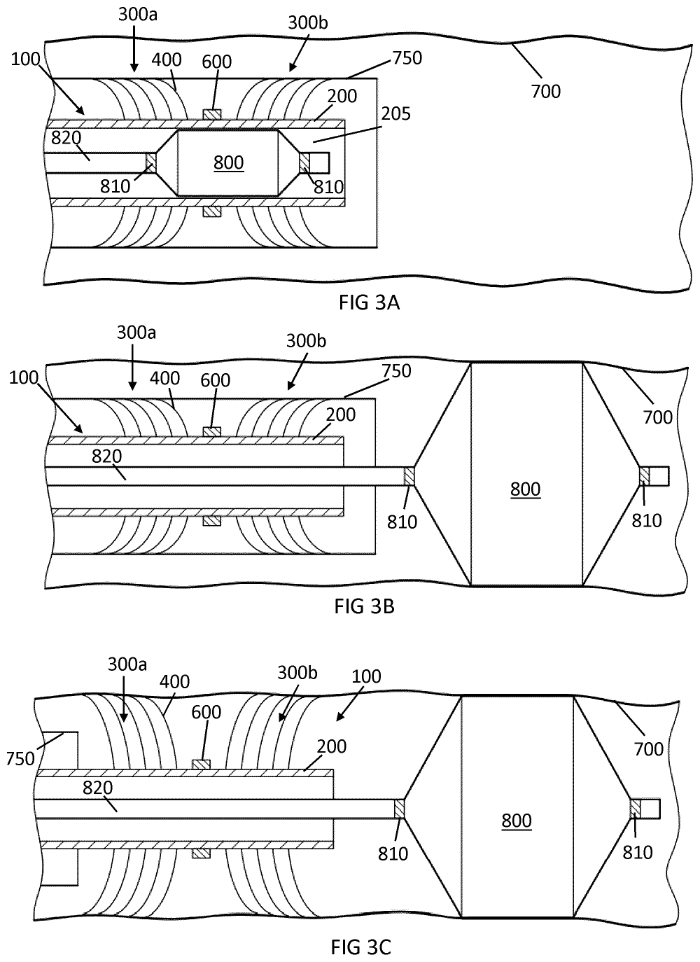

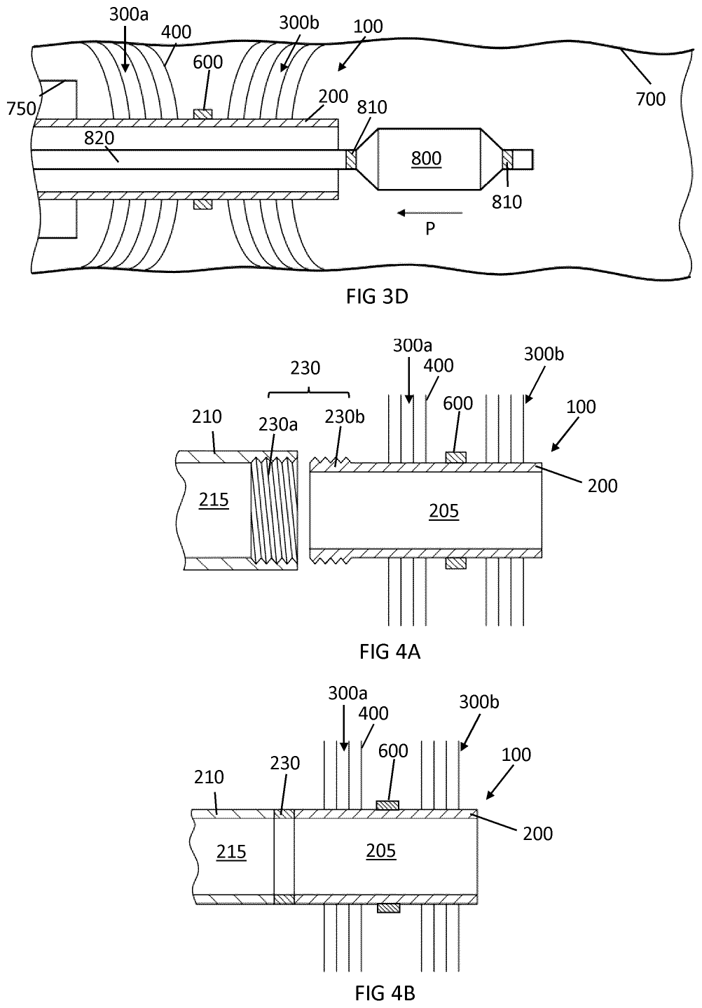

FIG. 3A shows an embolization system according to one or more embodiments. The embolization system comprises a self-expandable embolization device 100. In the illustrated embodiment, the self-expandable embolization device 100 is similar to the embolization device described with respect to FIG. 1B, and comprises a core 200 and a plurality of flexible bristles 400. The plurality of flexible bristles 400 may be comprised in a plurality of bristle segments 300a, 300b, as described with respect to FIG. 1A. The embolization device 100 may also comprise a radiopaque marker 600 disposed on the core 200 positioned in between the segment 300a, 300b. The radiopaque marker 60 may be located proximal or distal to the bristle segments 300a, 300b or the device 100 may comprises a plurality of radiopaque markers at a plurality of positions along the core 200.

The core 200 is a stem and defines an inner lumen 205 which is sized to fit an inflatable body 800 in a deflated configuration, which provides a more compact delivery configuration of the embolization system. The inflatable body may be made of, for example, a biocompatible polymer or similar. The inflatable body 800 may comprise radiopaque markers 810 at a proximal and/or a distal end of the inflatable body 800. The inflatable body 810 may be connected to an inflation tube for delivering a fluid to the inflatable body 800 to inflate it.

Whilst in FIGS. 3A to 3D the embolization device is similar to that shown in FIG. 1B, the embolization device 100 may instead be substantially similar to that shown in FIGS. 2A and 2B.

The embolization device 100 is removably connectable at a proximal end to a delivery element, as discussed in further detail with respect to FIGS. 4A and 4B.

In use, a delivery catheter 750 is positioned at a target location in a bodily lumen 700 such as a blood vessel. The embolization system comprising the embolization device 100 and the inflatable body 800 is delivered through the delivery catheter 750 to the distal end of the delivery catheter 750 (e.g. by pushing a delivery element connected to the embolization device 100 and the inflation tube 820 through the delivery catheter 750).

The inflatable body 800 is then moved distally relative to the embolization device 100 so that the inflatable body 800 is delivered to the bodily lumen from the delivery catheter 750 (e.g. by maintaining the delivery element connected to embolization device 100 and the delivery catheter 750 in position, and by pushing the inflation tube 820 distally relative to the delivery element and delivery catheter 750). As shown in FIG. 3B, the inflatable body 800 is then inflated by delivering a fluid to the inflatable body 800 via inflation tube 820 (for example a saline solution or similar biocompatible fluid), such that the inflatable body 800 is anchored to the bodily lumen 700. It may be verified that the inflatable body 800 is in the correct location (for example, a predetermined distance from a target site for the embolization device 100) by viewing the location of the inflatable body using radiography or similar, or viewing the location of the radiopaque markers 810.

Once the inflation body 800 is determined as being in the correct position, the embolization device 100 is delivered from the delivery catheter 750 by moving the embolization device 100 distally relative to the delivery catheter 750. For example, the embolization device 100 may be held at a predetermined distance from the inflatable body 800 inside the delivery catheter 750. The delivery element connected to the embolization device 100 may then be held or pinned in place, and the delivery catheter 750 may be retracted so that the embolization device 100 is deployed from the delivery catheter 750 as shown in FIG. 3C, so that the embolization device is anchored to the bodily lumen 700.

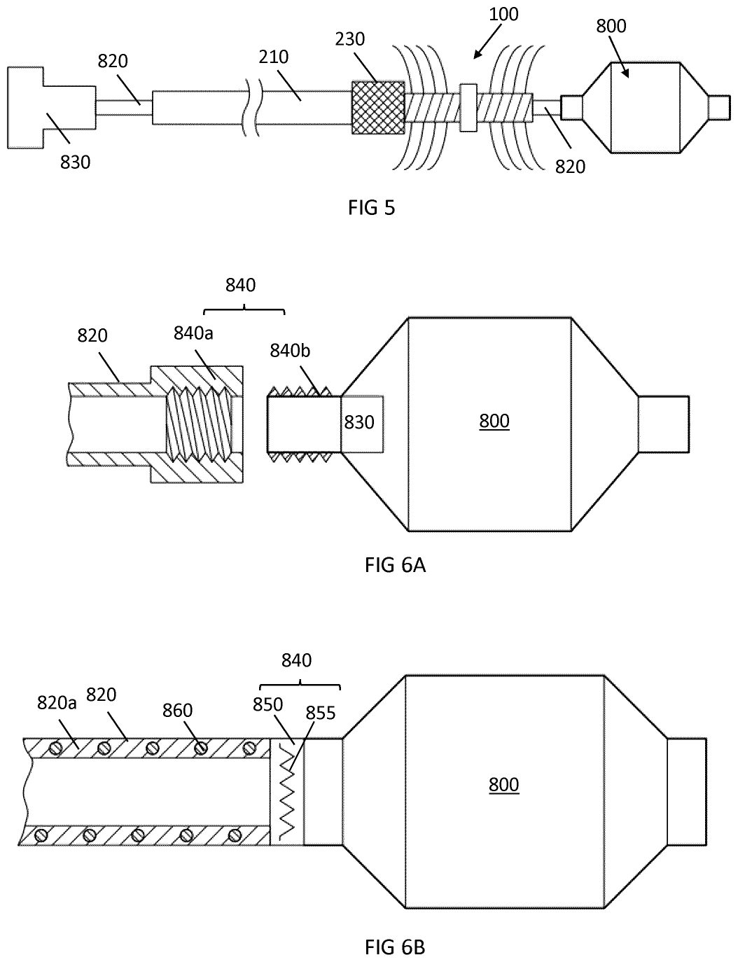

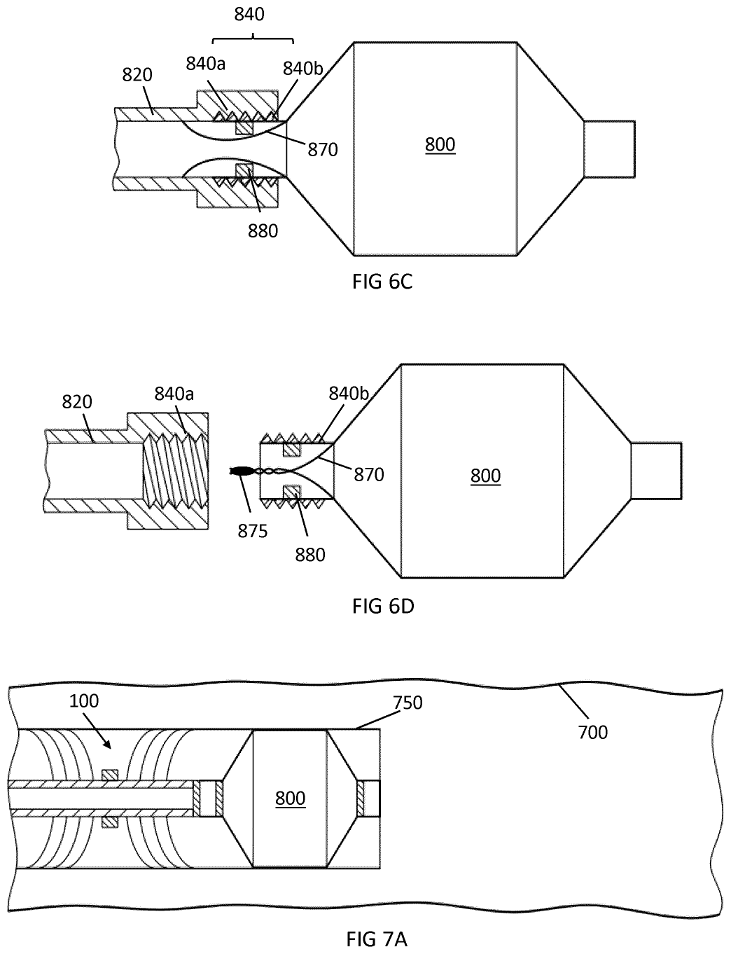

The inflatable body 800 may then be deflated and retracted in a proximal direction P, as shown in FIG. 3D. The delivery element connected to the embolization device 100 may be disconnected and retracted, as well as the delivery catheter 750, so that the embolization device 100 is deployed. In other embodiments, the inflation tube 820 may be connected to the inflatable body 800 by a detachable sealing mechanism (such as those described with reference to FIGS. 6A to 6D) which is configured to seal the inflatable body 800 when the inflation tube 820 detaches, so that the inflatable body 800 is also left implanted in the body lumen 700.

The use of the inflatable body 800 to pre-position the embolization system before the embolization device 100 is deployed (or even partially deployed) from the delivery catheter 750 allows the embolization system to be more easily re-positioned as needed by simply deflating, re-positioning and re-inflating the balloon, whilst the embolization device 100 remains in the collapsed configuration, before deploying the embolization device 100 in the correct location.

Whilst in the illustrated embodiments both the embolization device 100 and the inflatable body 800 comprise radiopaque markers, in some embodiments one or both devices do not comprise any radiopaque markers.

FIG. 4A shows a detach mechanism 230 for removably connecting the self-expandable embolization device 100 to a delivery element 210. The detach mechanism 230 comprises a male screw thread 230b on a proximal end of the embolization device 100 and a female screw thread 230a on a distal end of the delivery element 210 (the male screw thread may instead be located on the delivery element 210 and the female screw thread on the embolization device 100. Whilst FIG. 4A shows a self-expandable embolization device similar to that shown in FIG. 1B, the detach mechanisms disclosed herein may be used for any of the embolization devices disclosed herein. For example, a male or female screw thread could be attached to a proximal end of the devices shown in FIG. 1B or 2A and 2B, or the devices shown in FIG. 1B or 2A and 2B could be connected to a delivery element by an electrolytic element. The delivery element 210 may define a lumen 215 through which the inflation tube 820 is configured to extend. The inflation tube 820 and inflatable body 800 may be slidable relative to the lumen 215. When the male and female screw threads are connected, the delivery element 210 may be used to push the embolization device 100 through a delivery catheter (e.g. catheter 750). Once the embolization device 100 is deployed (for example as shown in FIG. 3D), the delivery element 210 may be rotated to unscrew the detach mechanism 230 and detach the delivery element 210 from the device 100.

FIG. 4B shows another detach mechanism 230 which may be used for any embolization device disclosed herein. The detach mechanism 230 comprises an electrolytic material which connects the delivery element 210 to the embolization device 100. The electrolytic material is operable to disintegrate by electrolysis in the bodily lumen upon application of an electric current to the electrolytic element. The electrolytic element may be formed of, for example, platinum, stainless steel, nitinol or cobalt chromium or an alloy thereof. The electrolytic element may be configured to disintegrate when a positive current of a particular current amplitude and voltage is applied for a predetermined amount of time. The electrolytic material may be electrically connectable to an external voltage or current source via an electrical wire (not shown) which extends along the length of the delivery element 210, or the delivery element 210 itself may be electrically conductive. Whilst FIG. 4B shows a self-expandable embolization device similar to that shown in FIG. 1B, the detach mechanisms disclosed herein may be used for any of the embolization devices disclosed herein. The delivery element 210 may define a lumen 215 through which the inflation tube 820 is configured to extend. The inflation tube 820 and inflatable body 800 may be slidable relative to the lumen 215. When the electrolytic material connects the delivery element 210 and the embolization device 100, the delivery element 210 may be used to push the embolization device 100 through a delivery catheter (e.g. catheter 750). Once the embolization device 100 is deployed (for example as shown in FIG. 3D), the electrical current may be applied to disintegrate the electrolytic material by electrolysis in the bodily lumen in order to detach the delivery element 210 from the device 100.

FIG. 5 shows a side view of the embolization system of FIGS. 4A to 4D. As previously disclosed, the inflation body 800 is positionable distal to the embolization device 100. The embolization device 100 comprises a lumen through which the inflation tube 820 extends. The inflation tube 820 is connected at a proximal end to a fluid port 830 through which fluid may be delivered to the inflatable body 800 via inflation tube 820. The embolization device 100 is deliverable to a distal end of a delivery catheter (e.g. catheter 750) by delivery element, which is a tubular body extending through the delivery catheter along its length. The inflation tube 820 and inflatable body 800 are slidable relative to the delivery element 210.

As disclosed with respect to FIGS. 3A to 3D, the balloon may be deflated and removed once the embolization device 100 is deployed in the body lumen. In other embodiments, the balloon may be detached from the inflation tube in the inflated configuration and left implanted in the body lumen.

For example, as shown in FIG. 6A, which shows a cross-sectional side view of a detachable sealing mechanism 840 for the inflatable body 800, the inflatable body 800 may comprise a one-way valve 830 which is configured to allow fluid into the inflatable body 800 and to inhibit the exit of fluid from the inflatable body 800. The inflatable body 800 may be connectable to the inflation tube 820 by a detach mechanism 840. The inflation tube 820 may comprise the female screw thread 840a and the inflation body 800 the male thread 840b, or vice versa. Such an inflatable body 800 may be used in any of the embodiments disclosed herein. In the embodiment shown in FIGS. 3A to 3D, the inflatable body 800 and the embolization device 100 may be deployed in the body lumen 700 as shown in FIG. 3C. Instead of deflating the inflatable body 800 and removing it as shown in FIG. 3D, the inflatable body 800 may be detached from the inflation tube 820 (for example by the mechanism shown in FIG. 6A) and left in the body lumen in the inflated configuration. The detach mechanism 840 may instead comprise an electrolytic element connecting the inflation tube 820 and the inflation body 800 similar to that shown in FIG. 4B.

In any of the embodiments disclosed herein, the outer surface of the inflatable body 800 may be coated with a thrombogenic agent such as prothrombogenic drug compounds or coagulation cascade factors (e.g. Fibrinogen, Prothrombin) to promote thrombosis in the body lumen.

FIG. 6B shows a cross-sectional side view of another possible detachable sealing mechanism 840 for detaching the inflation body 800 from the inflation tube 820 in the inflated configuration. In FIG. 6B, the detach mechanism 840 comprises a meltable material 850 (such as a meltable polymer) and a resistive element such as a resistive heater 855. Suitable meltable materials include polyimides, polyamides such as Grilamid, Nylon, Silicone or polyurethanes. The meltable material may have a melting temperature of between 230° F. to 450° F. (110° C. to 232° C.). The inflation tube 820 comprises a conductive wire 860 electrically connected to the resistive heater 855. The meltable material 850 comprises one or more holes through which fluid may pass in order to inflate inflatable body 800. Once the inflatable body 800 is inflated, an electric current may be applied to the conductive wire 860, causing the resistive heater 855 to heat and the meltable material 850 to melt. The holes in the meltable material 850 close and the inflation tube 820 is detached from the meltable material 850.

FIG. 6C shows a cross-sectional side view of another possible detachable sealing mechanism 840 for detaching the inflation body 800 from the inflation tube 820 in the inflated configuration. In FIG. 6C, the detach mechanism 840 comprises a male screw thread 840b on the inflatable body 800 and a female screw thread 840a on the inflation tube 820 (or vice-versa), and a twistable material 870. The twistable material 870 is connected to the inflatable body 800 at one longitudinal end, and to the inflation tube 820 at another longitudinal end. When the inflation tube 820 and the inflatable body 800 are connected as shown in FIG. 6C, fluid is deliverable from the inflation tube 820 to the inflatable body 800. Once the inflatable body 800 is inflated, the inflation tube 820 is twisted to unscrew the screw threads 840a, 840b. In doing so, the twistable material 870 is twisted until it detaches from the inflation tube 820 and a knot 875 is formed, inhibiting fluid from exiting the inflatable body 800, as shown in FIG. 6D. Further, when in the body lumen, a thrombosis formed rapidly around the knot 875 which ensures that the inflatable body 800 remains sealed and that the twistable material 875 does not unknot or untwist. A collar 880 may also be provided to reduce leakage from the inflatable body 800. The twistable material may be made of, for example, any suitable flexible material such as Grilamid, Silicone or polyurethane.

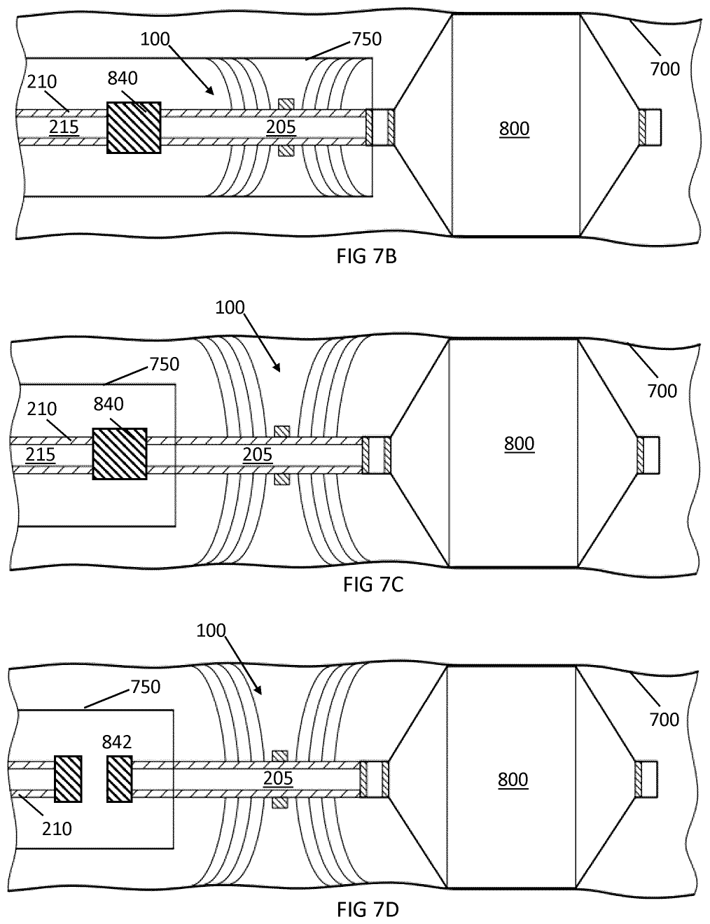

FIGS. 7A to 7D show a cross-sectional side view of another embolization system according to one or more embodiments. The embolization system shows an inflatable body 800 which is fixed distally to the embolization device 100. The embolization device 100 illustrated is similar to that shown in FIG. 1A, although any suitable embolization device (such as that shown in FIGS. 2A and 2B) may be used instead. The inflatable body 800 may comprise any of the features disclosed with respect to the other embodiments, such as the radiopaque markers. In the illustrated embodiment, the embolization device 100 and the inflatable body 800 are jointly pushed through the delivery catheter by a single delivery element 210 and are connected to the delivery element 210 by a single detach mechanism 840 which is actuatable to detach both the embolization device 100 and the inflatable body 800, and to seal the inflatable body upon detachment.

As shown in FIG. 7B, the delivery catheter 750 is positioned at a target site in the body lumen 700 and the inflatable body 800 is then delivered from the distal end of the delivery catheter 750 (for example by pinning the delivery element 210 and pulling the delivery catheter 750 in a proximal direction). The inflatable body 800 is inflated to anchor the inflatable body 800 to the body lumen 700. In the illustrated embodiment, the embolization device comprises a stem defining an inner lumen 205 and the delivery element 210 also defines an inner lumen 215. Collectively, the inner lumens 205 and 215 function as an inflation tube which fluidically connects the interior of the inflatable body 800 to a proximal end of the delivery element, through which the inflatable body 800 can be inflated. The lumens 205 and 215 are connected via a detach mechanism 840.

If the inflatable body 800 is incorrectly deployed in the body lumen 700, it is deflated by removing fluid via the lumens 205, 215, repositioned in the body lumen 70, and reinflated. Once it is determined that the inflatable body 800 is correctly positioned in the body lumen 700, the embolization device 100 is deployed (for example by pinning the delivery element 210 and retracting the delivery catheter 750). This configuration is shown in FIG. 7C.

The delivery element 210 is then detached from the embolization device 100 by actuating the detach mechanism 840. The detach mechanism 840 seals the inflatable body 800, indicated by sealing portion 842 in FIG. 7D. The delivery element 210 and delivery catheter 750 are then removed from the body lumen 700.

The detach mechanism 840 may comprise any of the detach mechanisms shown in FIGS. 6A to 6D. In the case of a screw mechanism like that shown in FIG. 6A, the one-way valve may be provided in the inflatable body 800 or the lumen 205.

In other embodiments, the detach mechanism 840 shown in FIGS. 7A to 7D may not be configured to seal the inflatable body and may correspond to the detach mechanism shown in FIG. 4A or 4B. In such embodiments, the inflatable body 800 is deflated once the embolization device 100 is deployed (such as in FIG. 7C), and the embolization device 100 and inflatable body 800 are detached from the delivery element 210 with the embolization device 100 in the deployed configuration and the inflatable body 800 in the deflated configuration.

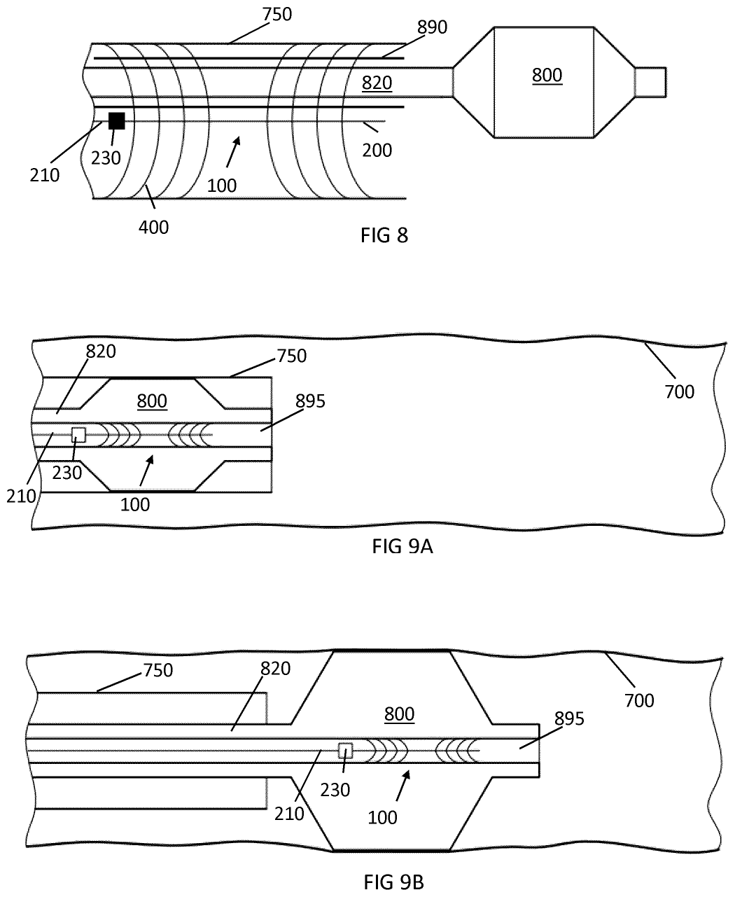

FIG. 8 shows a cross-sectional side view of another embolization system according to one or more embodiments. In the illustrated embodiment, the embolization device 100 is similar to that shown in FIG. 1B although it may instead be similar to that described with reference to FIG. 1A or 2A and 2B. The embolization system comprises an embolization device 100 which is removably connected to a delivery element 210 by a detach mechanism 230. The detach mechanism 230 may be, for example, a screw detach mechanism comprises a male and female screw thread on the delivery element 210 and embolization device 100 (similar to the mechanism of FIG. 4A), or may comprise an electrolytic element connecting the delivery element 210 and embolization device 100, configured to disintegrate by electrolysis upon application of an electric current (similar to the mechanism of FIG. 4B). The embolization system also comprises an inflatable body 800 which is inflatable via an inflation tube 820 which extends along the length of the delivery catheter 750. The inflatable body 800 and/or embolization device 100 may comprise one or more radiopaque markers. The inflation tube 820 and inflatable body 800 may be slidable or fixed relative to the delivery element 210 and embolization device 100. As in the previously-described embodiments, the delivery catheter 750 is advanced to a target location in a body lumen. The inflatable body 800 is delivered from the delivery catheter 750 (for example by pinning the inflation tube 820 and pulling the delivery catheter 750) and inflated to be anchored to the body lumen. The inflatable body 800 may be deflated, re-positioned and re-inflated if necessary. Once it is determined that the inflatable body 800 is correctly deployed in the body lumen, the embolization device 100 is deployed from the delivery catheter 750 (for example by pinning the delivery element 210 and pulling the delivery catheter 750). The detach mechanism 230 is then actuated to disconnect the delivery element 210 from the embolization device 100. Once the embolization device 100 is deployed, the inflatable body 800 may be deflated and removed from the body lumen along with the delivery element 210 and the delivery catheter 750. In other embodiments, the inflatable body 800 may also be detached from the inflation tube 820 (for example the inflatable body 800 may be connected to the inflation tube 820 by one of the detach mechanisms shown in FIGS. 6A to 6D). The embolization system may further comprise a lumen 890 which extends at least from a proximal end of the embolization device 100 to a distal end of the embolization device 100. The bypass tube allows the inflatable body 800 to be removed without contacting, for example, bristles or other features of the embolization device 100 upon removal, which may alter the deployed configuration of the embolization device 100 in the body lumen. The lumen 890 may be fixed to the embolization device 100 or slidable relative to the embolization device such that it may also be removed from the body lumen with the inflatable body 800.

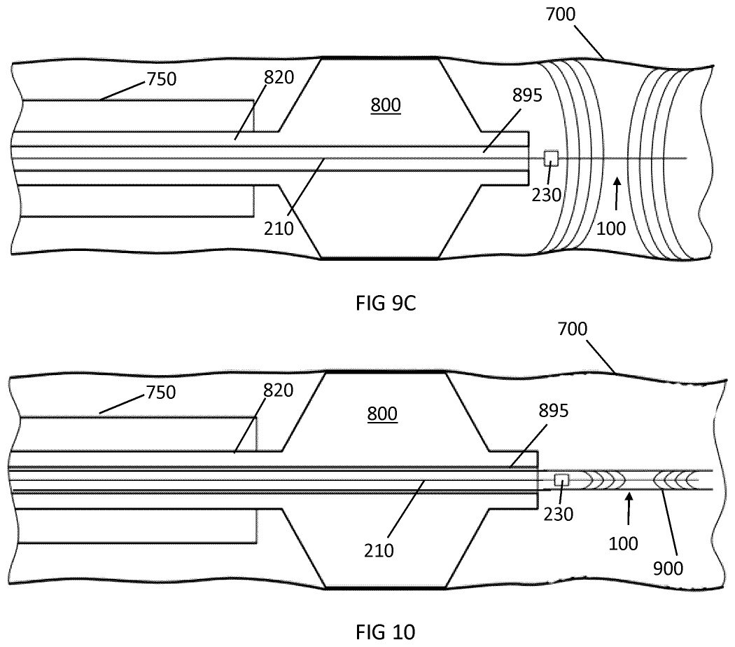

FIGS. 9A to 9C show a cross-sectional side view of an embolization system according to one or more embodiments. The embolization system comprises an inflatable body 800 connected to an inflation tube 820. The inflatable body 800 and inflation tube 820 comprises an inner lumen 895 configured to slidably receive a delivery element 210 for delivering the embolization device. The inner lumen 895 may be configured to receive an embolization device 100. The inflation tube 820 also comprises an inflation lumen (not shown) extending alongside the inner lumen 895 for inflating the inflatable body 800. The embolization device 100 shown in similar to that shown in FIG. 1A, whereas in some embodiments the embolization device 100 may be similar to the other embolization devices described with reference to FIGS. 1B, 2A and 2B. The embolization device 100 is connected to a delivery element 210 via a detach mechanism 230, which may be similar to those shown in FIGS. 6A and 6B. The delivery element 210 extends through the inner lumen 895. The delivery element 210 and embolization device 100 are slidable relative to the inflatable body 800 and the inflation tube 820. The inflatable body 800 and the embolization device 100 are pushed through the delivery catheter 750 to a distal end of the delivery catheter 750 by advancing the delivery element 210 and inflation tube 820 through the delivery catheter 750.

The inflatable body 800 is deployed from the delivery catheter 750 (for example by pinning the inflation tube 820 and pulling the delivery catheter 750) and inflated by delivering fluid to the inflatable body 800 via inflation tube 820. This configuration is shown in FIG. 9B. The inflatable body 800 may be deflated, re-positioned and re-inflated if it is deployed incorrectly.

Once the inflatable body 800 is correctly deployed, the embolization device 100 is deployed from the inflatable body 800 (for example by pinning the inflation tube 820 and advancing the delivery element 210). This configuration is shown in FIG. 9C. The embolization device 100 may then be detached by actuating the detach mechanism 230. The inflatable body 800, delivery element 210 and delivery catheter 820 are then removed from the body lumen.

In other embodiments, the inflatable body 800 may be detachable from the inflation tube 820 via a mechanism similar to one of the mechanisms shown in FIGS. 6A to 6D and the inflatable body 800 may also be left in the body lumen in an inflated configuration.

FIG. 10 shows a cross-sectional side view of another embolization system according to one or more embodiments. The embolization system comprises an inflatable body 800 connected to an inflation tube 820. The inflatable body 800 and inflation tube 820 comprise an inner lumen 895 configured to receive an embolization device 100. The inflation tube 820 also comprises an inflation lumen (not shown) extending alongside the inner lumen 895 for inflating the inflatable body 800. The embolization device 100 shown is similar to that shown in FIG. 1A, whereas in some embodiments the embolization device 100 may be similar to the other embolization devices described with reference to FIGS. 1, 2A and 2B. The embolization device 100 is connected to a delivery element 210 via a detach mechanism 230, which may be similar to those shown in FIGS. 6A and 6B. The delivery element 210 extends through the inner lumen 895. The delivery element 210 and embolization device 100 are slidable relative to the inflatable body 800 and the inflation tube 820. In other embodiments, the delivery element 210 and embolization device 100 may be fixed relative to the inflatable body 800 in a position distal to the embolization device 100, such as the position shown in FIG. 10. The embolization system further comprises a sheath 900 extending through the lumen 895 and containing the delivery element and the embolization device 100. The inflatable body 800 and the embolization device 100 are pushed through the delivery catheter 750 to a distal end of the delivery catheter 750 by advancing the delivery element 210 and inflation tube 820 through the delivery catheter 750.

The inflatable body 800 is deployed from the delivery catheter 750 (for example by pinning the inflation tube 820 and pulling the delivery catheter 750) and inflated by delivering fluid to the inflatable body 800 via inflation tube 820. The inflatable body 800 may be deflated, re-positioned and re-inflated if it is deployed incorrectly.

Once the inflatable body 800 is correctly deployed, the embolization device 100 is deployed from the sheath 900 (for example by pinning the delivery element 210 and pulling the sheath 900 in a proximal direction). The embolization device 100 may then be detached by actuating the detach mechanism 230. The inflatable body 800, delivery element 210, sheath 900 and delivery catheter 820 are removed from the body lumen.

In other embodiments, the inflatable body 800 may be detachable from the inflation tube 820 via a mechanism similar to one of the mechanisms shown in FIGS. 6A to 6D and the inflatable body 800 may also be left in the body lumen in an inflated configuration.

In all of the embodiments disclosed herein, the embolization device 100 may be slidably or non-slidably connected to the inflatable body 800. For example, in embodiments where the embolization device 100 is delivered by pushing a delivery element 210 and the inflatable body 800 is delivered by pushing inflation tube 800, the delivery element 210 and inflation tube 800 may have a fixed connection to each other and may be slidable in unison relative to the delivery catheter 750 (in such embodiments the inflatable body 800 is in a fixed position relative to the embolization device 100 away from the embolization device 100 such that it can be inflated whilst the embolization device 100 remains in the collapsed delivery configuration).

The embolization systems disclosed herein may be manufactured by providing an embolization device, providing an inflatable body, and connecting the embolization device to the inflatable body in a position such that the inflatable body is inflatable from the deflated configuration to the inflated configuration as the self-expandable embolization device remains in the collapsed delivery configuration. For example, the embolization device and inflatable body may be slidably connected (for example by inserting an inflation tube for inflating the inflatable body into a device lumen of the embolization device, or by inserting the embolization device and a delivery element connected to the embolization device into an inner lumen of the inflatable body), or the embolization device may be fixed to the inflatable body.

All of the above are fully within the scope of the present disclosure, and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. An embolization system for promoting clot formation in a body lumen, comprising:
a self-expandable embolization device, having a collapsed delivery configuration and an expanded deployed configuration in which the self-expandable embolization device is radially expanded to anchor to a bodily lumen; and
an inflatable body connected to the embolization device, the inflatable body having a deflated configuration and an inflated configuration, wherein in the inflated configuration the inflatable body anchors to the bodily lumen;
wherein the inflatable body is positionable relative to the self-expandable embolization device such that it is inflatable from the deflated configuration to the inflated configuration as the self-expandable embolization device remains in the collapsed delivery configuration.

2. The embolization system according to claim 1, wherein the self-expandable embolization device comprises a core and a plurality of flexible bristles extending at least radially outwardly from the core, the flexible bristles having a collapsed delivery configuration and an expanded deployed configuration in which the bristles extend at least radially outwardly from the core to anchor the embolization device in a bodily lumen.

3. The embolization system according to claim 1, wherein the self-expandable embolization device comprises a self-expandable layer, the self-expandable layer comprising a plurality of braided strands, the plurality of braided strands having a collapsed delivery configuration and an expanded deployed configuration in which a plurality of the braided strands anchor the embolization device in a bodily lumen, and in which a plurality of the braided strands extend across the bodily lumen to occlude the bodily lumen.

4. The embolization system according to claim 1, wherein the embolization device and the inflatable body are slidably connected or non-slidably connected to one another.

5. The embolization system according to claim 1, comprising a sealing mechanism actuatable to fluidically seal the inflatable body in the inflated configuration.

6. The embolization system according to claim 5, wherein the sealing mechanism comprises a one-way valve configured to prevent fluid from exiting the inflatable body.

7. The embolization system according to claim 5, wherein the sealing mechanism comprises a resistive element and a meltable material, wherein an electrical current applied to the resistive element melts the meltable material to seal an inflation port of the inflatable body.

8. The embolization system according to claim 5, wherein the sealing mechanism comprises a flexible tubular material having an open configuration in which fluid may enter the balloon via a lumen defined by the flexible tubular material, wherein the flexible tubular material is twistable into a closed configuration in which the inflatable body is fluidically sealed.

9. The embolization system according to claim 1, wherein the embolization device is configured to be connected to a delivery element via a detach mechanism, the delivery element for delivering the embolization device through a delivery catheter.

10. The embolization system according to claim 9, wherein the detach mechanism comprises a male or female screw thread on the embolization device or the detach mechanism comprises an electrolytic element connecting the embolization device to the delivery element.

11. The embolization system according to claim 1, wherein the inflatable body is fixed relative to the embolization device and the embolization system is configured to be connected to a single delivery element via a detach mechanism, the delivery element for delivering the embolization device and the inflatable body through a delivery catheter.

12. The embolization system according to claim 11, wherein the detach mechanism includes a sealing mechanism actuatable to fluidically seal the balloon in the inflated configuration.

13. The embolization system according to claim 11, wherein the embolization device comprises a stem and a plurality of flexible bristles secured to the stem and extending at least radially outwards from the stem, and wherein the stem defines an inner lumen fluidically connected to the interior of the inflatable body.

14. The embolization system according to claim 1, wherein the embolization device comprises a device lumen extending longitudinally through the embolization device, wherein the device lumen receives an inflation tube for inflating the inflatable body;

15. The embolization system of claim 14, wherein the embolization device and the inflatable body are slidably connected to one another and the inflatable body is configured to fit partially or wholly within the device lumen in the deflated configuration.

16. The embolization system according to claim 1, wherein the inflatable body comprises an inner lumen extending longitudinally through the inflatable body and configured to receive a delivery element for delivering the embolization device.

17. The embolization system according to claim 16, further comprising a sheath slidably received in the inner lumen, wherein the embolization device fits inside the sheath in the collapsed delivery configuration.

18. The embolization system according to claim 1, wherein the inflatable body comprises one or more radiopaque markers, and/or the embolization device comprises one or more radiopaque markers.
